(19) United States
(12) Patent Application Publication
(10) Pub. No.: US 2026/0295141 A1
(43) Pub. Date: Oct. 1, 2026

(54) DUAL-FLOW BIOLOGICAL FLUID EXCHANGE DEVICE

(71) Applicant: University of Kansas, Lawrence, KS (US)

(72) Inventor: Travis Harrison Kimple, Garden City, KS (US)

(21) Appl. No.: 19/576,284

(22) Filed: Mar. 24, 2026

Related U.S. Application Data
(60) Provisional application No. 63/777,285, filed on Mar. 25, 2025.

(51) Int. Cl. A61M 1/00; A61F 9/00; A61M 5/32

(52) U.S. Cl. CPC A61M 1/774; A61F 9/0017; A61M 5/3295

(57) ABSTRACT

In some embodiments, a fluid-exchange system includes a dual-flow body having a first fluid passage and a second fluid passage, a first needle, a second needle, a first fluid reservoir, and a second fluid reservoir. In some embodiments, the first fluid passage is configured to deliver a replacement fluid to a surgical site while the second fluid passage is configured to withdraw an anatomical fluid from the surgical site. A cap may be connected to one of the fluid reservoirs and may include one or more vent features configured to permit air ingress while reducing undesired fluid leakage, thereby facilitating passive delivery of the replacement fluid. In some embodiments, the system may be utilized for anterior chamber paracentesis or another procedure involving coordinated fluid withdrawal and fluid delivery.

### CROSS-REFERENCE TO RELATED APPLICATIONS

This application claims priority to and the benefit of United States Provisional Patent Application Serial No. 63/777,285, filed Mar. 25, 2025, which is hereby incorporated by reference in its entirety.

### BACKGROUND

In some medical procedures, it may be desirable to withdraw a fluid from a surgical site while managing pressure and/or fluid volume at the surgical site. In some cases, withdrawal of the fluid without compensating fluid delivery may result in undesirable pressure changes, collapse of a target chamber, reduced procedural control, and/or increased risk of tissue disruption. Accordingly, a system that facilitates coordinated fluid withdrawal and fluid delivery in a controlled manner may be desirable.

### SUMMARY

In some embodiments, a fluid-exchange device for a medical procedure may include a dual-flow body that supports two different fluid passages through a common device body. A first fluid passage may extend from a first fluid inlet to a first fluid outlet, and a second fluid passage may extend from a second fluid inlet to a second fluid outlet. The first fluid inlet may be oriented at an angle relative to the second fluid outlet such that the two passages have different geometries and may support different component orientations. A first needle may be connected to the first fluid outlet and extend from the first fluid passage, and a second needle may be connected to the second fluid inlet and extend from the second fluid passage, such that the device may be positioned at a surgical site for coordinated fluid exchange.

In some embodiments, a fluid-exchange system for a medical procedure may include a dual-flow body in combination with a first fluid reservoir and a second fluid reservoir connected thereto. The dual-flow body may include a first fluid passage associated with a first needle and a second fluid passage associated with a second needle. In some cases, the first fluid reservoir may be in fluid communication with the first fluid passage and the second fluid reservoir may be in fluid communication with the second fluid passage, with the first fluid reservoir oriented at an angle with respect to the second fluid reservoir. A first flow path may extend from the first fluid reservoir, through the first fluid passage, and out of the first needle, while a second flow path may extend from the second needle, through the second fluid passage, and into the second fluid reservoir. In this way, the system may support coordinated fluid delivery and fluid withdrawal through a common dual-flow body.

In some embodiments, a method of performing a medical procedure includes positioning a fluid-exchange device at a surgical site, where the fluid-exchange device includes a first needle and a second needle each connected to and extending from a dual-flow body. The first needle and the second needle may be used to penetrate the surgical site such that both needles are in fluid communication with an anatomical fluid at the surgical site. A portion of the anatomical fluid may then be withdrawn through the second needle via a second flow path extending from the second needle, through the dual-flow body, and into a second fluid reservoir connected to the dual-flow body. While that anatomical fluid is being withdrawn, a replacement fluid may be administered to the surgical site through the first needle via a first flow path extending from a first fluid reservoir connected to the dual-flow body, through the dual-flow body, and out of the first needle. In some cases, this coordinated withdrawal, and administration may facilitate controlled fluid exchange at the surgical site.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages will be set forth in the description that follows. Features and advantages of the disclosure may be realized and obtained by means of the systems and methods that are particularly pointed out in the appended claims. Features of the present disclosure will become more fully apparent from the following description and appended claims or may be learned by the practice of the disclosed subject matter as set forth hereinafter.

### BRIEF DESCRIPTION OF THE DRAWINGS

In order to describe the manner in which the above-recited and other features of the disclosure can be obtained, a more particular description will be rendered by reference to specific embodiments thereof which are illustrated in the appended drawings. For better understanding, the like elements have been designated by like reference numbers throughout the various accompanying figures. Understanding that the drawings depict some example embodiments, the embodiments will be described and explained with additional specificity and detail through the use of the accompanying drawings in which:

FIG. 1-1 illustrates a top view, FIG. 1-2 illustrates a first side view, and FIG. 1-3 illustrates a second side view of a fluid-exchange device, according to at least one embodiment of the present disclosure;

FIG. 2-1 illustrates a first side view of a fluid-exchange system, and FIGS. 2-2 and 2-3 illustrate second side views of the fluid-exchange system, according to at least one embodiment of the present disclosure;

FIG. 3-1 illustrates a side view of a cap, and FIGS. 3-2 and 3-3 illustrate top views of the cap, according to at least one embodiment of the present disclosure;

FIG. 4 illustrates an anatomical diagram of a human eye, according to at least one embodiment of the present disclosure; and

FIG. 5 illustrates a flow diagram for a method or a series of acts for performing a medical procedure, according to at least one embodiment of the present disclosure.

### DETAILED DESCRIPTION

This disclosure is generally related to controlled fluid exchange at a surgical site with fluid-exchange systems and devices. In some embodiments, a fluid-exchange device may be utilized to withdraw an anatomical fluid from the surgical site while also delivering a replacement fluid to the surgical site in a coordinated manner. In some cases, such coordinated fluid exchange may improve procedural control, reduce undesirable pressure changes at the surgical site, and/or facilitate withdrawal of a desired fluid volume.

In some embodiments, the systems described herein may include a dual-flow body configured with a first fluid passage for providing a first fluid path and a second fluid passage for providing a second fluid path. The dual-flow body may support a first needle and a second needle positioned with respect to one another such that the needles may be inserted into a surgical site in a coordinated manner. In some embodiments, the needles may be arranged generally parallel to one another. In various implementations, one side of the dual-flow body may be associated with withdrawal of an anatomical fluid and another side may be associated with administration of a replacement fluid. In some embodiments, the system may further include a first fluid reservoir and a second fluid reservoir connected to the dual-flow body. For example, one fluid reservoir may contain the replacement fluid for delivery through one flow path, while another fluid reservoir may receive the anatomical fluid through the other flow path. In some cases, delivery of the replacement fluid may occur passively. In some embodiments, a cap may be removably connected to one of the fluid reservoirs, such as to reduce spillage and/or leakage while still permitting venting and passive delivery without creating an undesired vacuum within the fluid reservoir.

In some embodiments, the fluid-exchange systems and devices described herein may be particularly useful for performing anterior chamber paracentesis of a human eye. For example, the anterior chamber of the human eye is normally filled with about 0.25 mL of aqueous humor. The anterior chamber is bounded anteriorly by the cornea and posteriorly by the iris, lens, and angle structures, and anterior chamber paracentesis generally refers to withdrawal of aqueous humor or another fluid from the anterior chamber. In various implementations, anterior chamber paracentesis may be performed as part of another ophthalmic procedure or as a standalone minor procedure, such as in an office setting. In some embodiments, one indication for anterior chamber paracentesis may be elevated intraocular pressure, such as may occur in connection with trauma, glaucoma, or prior surgery. In some embodiments, another indication may be diagnostic anterior chamber paracentesis, in which aqueous humor is withdrawn for laboratory analysis, such as polymerase chain reaction, microbiological culture, cytological analysis, biomarker analysis, or another diagnostic evaluation. In some cases, such testing may assist in diagnosis and management of inflammatory and/or other ocular conditions.

In some embodiments, anterior chamber paracentesis may be performed using a single small-gauge medical needle attached to a syringe. For example, a 27 gauge or 30 gauge needle attached to a 1 mL syringe may be utilized in some conventional procedures. In some cases, the eye may be anesthetized with a topical anesthetic and prepared with a topical antiseptic, such as dilute povidone iodine. In various implementations, the patient may be positioned supine or upright, with or without magnifying optics such as a slit lamp, loupes, or a surgical microscope. An eyelid speculum may be placed to reduce contact with the eyelids during the procedure. In some cases, the eye may be stabilized with a second instrument, such as forceps, while the physician inserts the needle into the anterior chamber through a peripheral region of the cornea. The plunger of the syringe may then be withdrawn until a desired amount of aqueous humor is removed, after which the needle may be withdrawn from the eye.

In some embodiments, such a procedure may be associated with a number of challenges, risks, and other shortcomings. For example, the anterior chamber is a small, closed, and non-rigid compartment bordered by delicate intraocular structures, such as the cornea, iris, and lens. In some cases, inadvertent injury to one or more of these tissues may occur during insertion, aspiration, withdrawal, or another stage of the procedure. In various implementations, withdrawal of an excessive amount of aqueous humor may cause intraocular pressure to decline to undesirably low levels, which may result in shallowing and/or collapse of the anterior chamber and may damage adjacent tissues or cause secondary injury to posterior ocular tissues. In some cases, diagnostic testing may require a minimum sample quantity, which may lead a physician to withdraw a relatively large amount of aqueous humor, such as up to about 0.2 mL. The desire to obtain a larger sample may increase procedural risk and may discourage performance of diagnostic anterior chamber paracentesis.

In some embodiments, the fluid-exchange systems and devices described herein may mitigate one or more of these challenges associated with anterior chamber paracentesis. For example, the systems and devices described herein may permit coordinated withdrawal of aqueous humor and administration of a replacement fluid. In some cases, this may reduce the risk of excessively low intraocular pressure during the procedure. In various implementations, the likelihood of anterior chamber shallowing and/or collapse may be reduced. In some embodiments, the systems and devices described herein may improve needle stability and procedural control during insertion and aspiration. In some cases, the systems and devices described herein may permit a greater amount of fluid to be safely withdrawn, which may improve the likelihood of obtaining a sample sufficient for diagnostic testing.

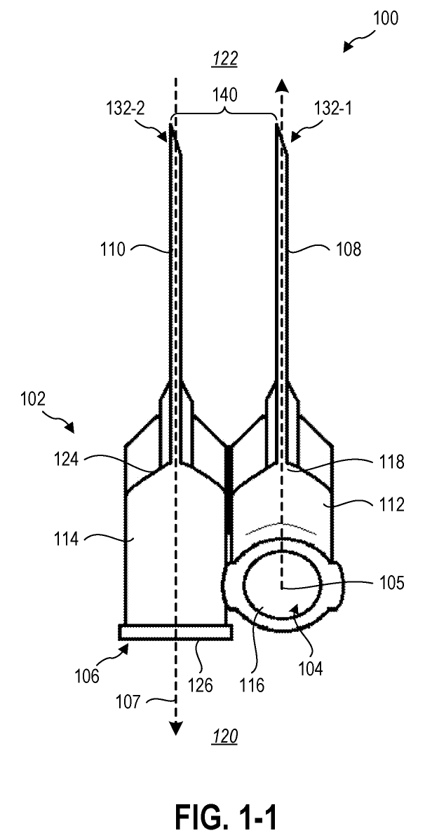

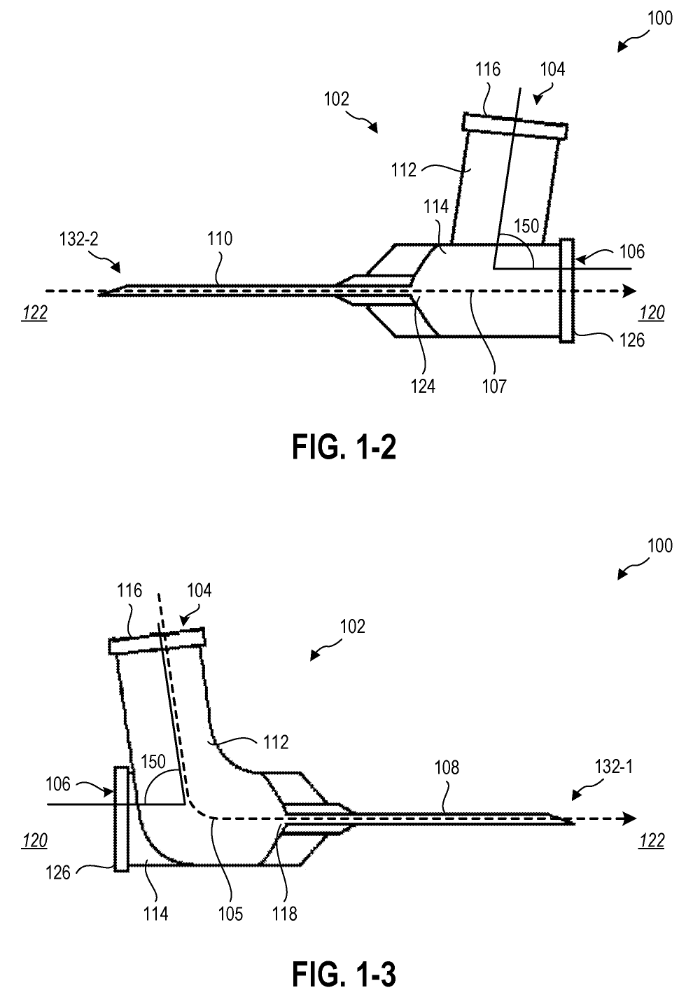

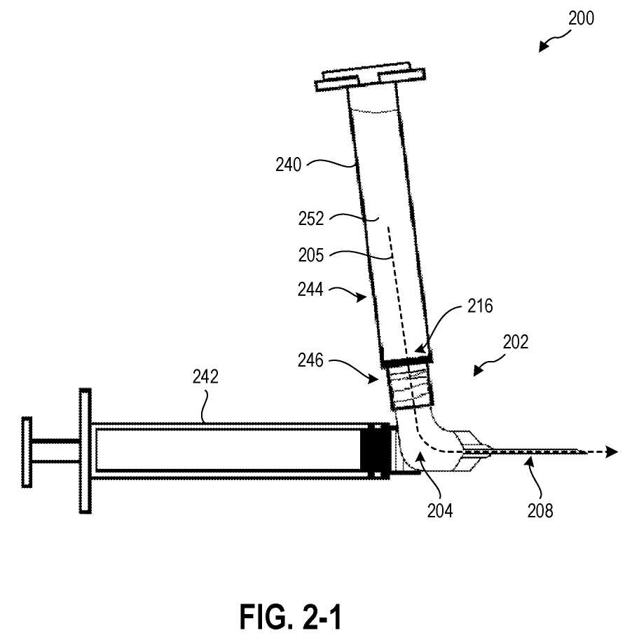

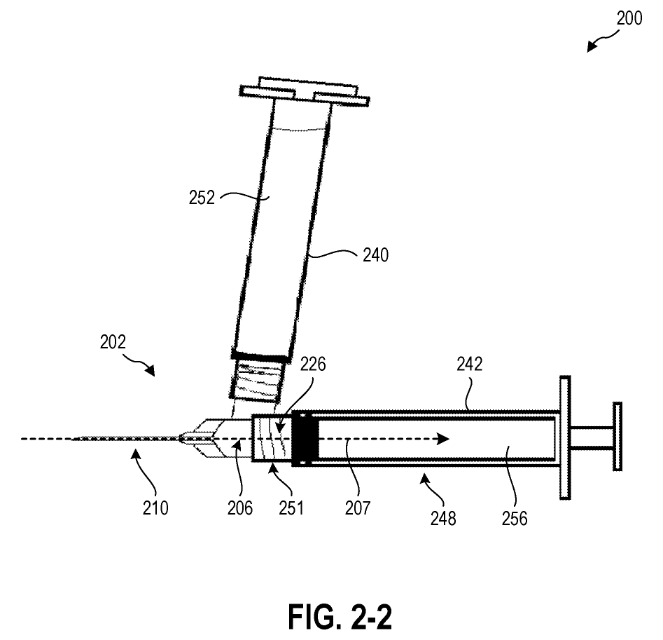

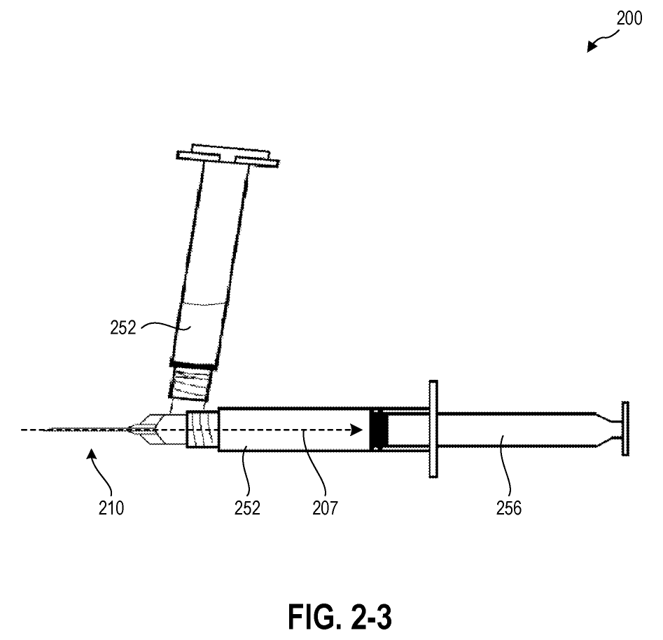

FIG. 1-1 illustrates a top view, FIG. 1-2 illustrates a first side view, and FIG. 1-3 illustrates a second side view of a fluid-exchange device 100, according to at least one embodiment of the present disclosure. In some embodiments, the fluid-exchange device 100 may be a device that may be utilized for various medical procedures, such as an anterior chamber paracentesis. For example, the fluid-exchange device 100 may be utilized to remove an anatomical fluid from a surgical site and, in combination, administer a replacement fluid to the surgical site.

In some embodiments, the fluid-exchange device 100 may include a dual-flow body 102. For example, the dual-flow body 102 may be a structure for connecting to one or more needles and/or one or more fluid reservoirs and for facilitating the influx and efflux of fluids through the dual-flow body 102. In some implementations, the dual-flow body 102 may define a first fluid passage 104 and a second fluid passage 106 through which different fluids and/or fluid flows (of the same or different fluid) may flow during a medical procedure. In some embodiments, the fluid-exchange device 100 may further define a first flow path 105 and a second flow path 107. For instance, the first fluid passage 104 may define at least a portion of the first flow path 105 through which a replacement fluid may flow toward a surgical site, and the second fluid passage 106 may define at least a portion of the second flow path 107 through which an anatomical fluid may flow away from the surgical site. In various embodiments, the dual-flow body 102 may also position a first needle 108 and a second needle 110 with respect to one another such that the first needle 108 and the second needle 110 may be inserted into the surgical site in a coordinated manner.

In some embodiments, the dual-flow body 102 may include a first body portion 112 and a second body portion 114. For example, the first body portion 112 may be associated with the first fluid passage 104 and the first flow path 105, and the second body portion 114 may be associated with the second fluid passage 106 and the second flow path 107. In some cases, the first body portion 112 and the second body portion 114 may be separate body portions that may be fixed, connected, joined, bonded, fused, welded, or otherwise secured together. In other implementations, the first body portion 112 and the second body portion 114 may be formed as a unitary, integral, and/or continuous body. For instance, the dual-flow body 102 may be formed as an integral body that includes both the first body portion 112 and the second body portion 114. In some embodiments, the first body portion 112 and the second body portion 114 may be arranged side-by-side such that the dual-flow body 102 positions the first needle 108 and the second needle 110 during insertion and use.

In some embodiments, the first fluid passage 104 may be formed in the dual-flow body 102. For example, the first fluid passage 104 may be formed in the first body portion 112. In some cases, the first fluid passage 104 may be a conduit, channel, lumen, bore, passage, or other pathway through which fluid may flow through the dual-flow body 102. In some implementations, the first fluid passage 104 may be defined from a first fluid inlet 116 to a first fluid outlet 118. For instance, the first fluid inlet 116 may be located at, near, facing, or adjacent to a first side 120 of the dual-flow body 102, and the first fluid outlet 118 may be located at, near, facing, or adjacent to a second side 122 of the dual-flow body 102. In some embodiments, the first flow path 105 may extend through the first fluid passage 104 between the first fluid inlet 116 and the first fluid outlet 118. In some cases, the first fluid inlet 116 and the first fluid outlet 118 may be oriented at an angle with respect to one another. For example, the first fluid passage 104 may extend non-linearly through at least a portion of the dual-flow body 102. In various embodiments, the first fluid passage 104 may be bent, angled, curved, redirected, or otherwise non-linear between the first fluid inlet 116 and the first fluid outlet 118. In some implementations, the non-linear configuration of the first fluid passage 104 may facilitate positioning a connected fluid reservoir at an upward or oblique angle relative to another portion of the fluid-exchange device 100, such as relative to another fluid reservoir connected to a second fluid outlet 126.

In some embodiments, the second fluid passage 106 may be formed in the dual-flow body 102. For example, the second fluid passage 106 may be formed in the second body portion 114. In some cases, the second fluid passage 106 may be a conduit, channel, lumen, bore, passage, or other pathway through which fluid may flow through the dual-flow body 102. In some implementations, the second fluid passage 106 may be defined from a second fluid inlet 124 to a second fluid outlet 126. For instance, the second fluid inlet 124 may be located at, near, facing, or adjacent to the second side 122 of the dual-flow body 102, and the second fluid outlet 126 may be located at, near, facing, or adjacent to the first side 120 of the dual-flow body 102. In some embodiments, the second flow path 107 may extend through the second fluid passage 106 between the second fluid inlet 124 and the second fluid outlet 126. In some embodiments, the first fluid inlet 116 and the second fluid outlet 126 may be considered to be on the same side of the dual-flow body 102, such as on the first side 120. In some embodiments, the first fluid outlet 118 and the second fluid inlet 124 may be considered to be on the same side of the dual-flow body 102, such as on the second side 122. In some embodiments, the second fluid inlet 124 and the second fluid outlet 126 may be oriented generally opposite one another. For example, the second fluid passage 106 may extend approximately linearly through at least a portion of the dual-flow body 102. In various embodiments, the second fluid passage 106 may define an approximately straight or linear flow path through the second body portion 114, for example, as compared with the first fluid passage 104.

In some embodiments, the first fluid passage 104 and the second fluid passage 106 may have different geometries. For example, the first fluid passage 104 may extend in a non-linear manner through the dual-flow body 102, while the second fluid passage 106 may extend in a relatively straight manner through the dual-flow body 102. In some cases, the differing geometries of the first fluid passage 104 and the second fluid passage 106 may facilitate positioning connected components in different orientations relative to one another. For instance, a first fluid reservoir connected to the first fluid inlet 116 may be oriented differently than a second fluid reservoir connected to the second fluid outlet 126. In some implementations, this difference in geometry may reduce crowding, facilitate reservoir placement, improve visibility at the surgical site, or support a desired gravity-assisted reservoir orientation, and combinations thereof. In some embodiments, the geometry of the first fluid passage 104 may be different than that shown. For example, the first fluid passage 104 may extend in a relatively straight manner in some implementations. In some cases, the first fluid passage 104 and the second fluid passage 106 may have the same or similar geometry such that a first fluid reservoir connected to the first fluid passage 104 and a second fluid reservoir connected to the second fluid passage 106 may extend, be positioned, be angled, or have other geometric characteristics that are the same or similar (and combinations thereof).

As described, the fluid-exchange device 100 may include the first needle 108 and the second needle 110. For example, the first needle 108 may be connected to the first fluid outlet 118 of the dual-flow body 102, and the second needle 110 may be connected to the second fluid inlet 124 of the dual-flow body 102. In some cases, the first needle 108 may be associated with administration of a replacement fluid to the surgical site, and the second needle 110 may be associated with removal of an anatomical fluid from the surgical site. In various implementations, the first needle 108 and the second needle 110 may be fixed and/or stabilized relative to the dual-flow body 102 such that movement and/or positioning of the dual-flow body 102 causes corresponding movement and/or positioning of the first needle 108 and the second needle 110.

In some embodiments, the first needle 108 and the second needle 110 may each include a tip, a shaft, and a lumen. For example, the first needle 108 may include a first tip 132-1, and the second needle 110 may include a second tip 132-2 (collectively tips 132). In some embodiments, the tips 132 may be beveled, sharpened, or otherwise configured to penetrate tissue at a surgical site. In some cases, the needle lumens may provide fluid communication between the surgical site and a corresponding one of the first fluid passage 104 and the second fluid passage 106. In some embodiments, the first needle 108 and the second needle 110 may be connected, fixed, attached, bonded, molded, or otherwise secured to the dual-flow body 102. In other implementations, the first needle 108 and the second needle 110 may be connectable to the dual-flow body 102. For example, in at least one embodiment, the dual-flow body 102 may include a first needle connector at the first fluid outlet 118 and a second needle connector at the second fluid inlet 124. In some cases, the first needle 108 may be part of a first needle hub removably connectable to the first needle connector, and the second needle 110 may be part of a second needle hub removably connectable to the second needle connector. A needle hub may be a medical connector structure configured to support and retain a needle cannula and to provide a fluid interface between the needle cannula and another medical component, such as a syringe, tubing, adapter, or another connector. For example, a needle hub may be a needle hub as is known and used in the medical industry. In this manner, the first needle connector and the second needle connector may be or may include threaded connectors, friction-fit connectors, slip-fit connectors, Luer-lock connectors, or other suitable needle connectors as are utilized in the medical industry for connecting needle hub to other medical devices and/or components.

In some embodiments, the first needle 108 and the second needle 110 may be medical-grade hypodermic needles. For example, the first needle 108 and the second needle 110 may be formed of stainless steel or another suitable biocompatible material. In some implementations, the first needle 108 and the second needle 110 may have the same gauge. In other implementations, the first needle 108 and the second needle 110 may have different gauges. For instance, one or both of the needles 108, 110 may have a gauge selected based on a desired procedure, a target surgical site, a desired flow rate, or another functional consideration. In some cases, one or both of the needles 108, 110 may have a gauge between about 25 gauge and about 30 gauge. For example, a 27 gauge needle may be utilized in one or more embodiments, such as for anterior chamber paracentesis. In some embodiments, a 30 gauge needle may be utilized, such as in embodiments where a smaller needle size is desirable for the procedure. Needle gauges larger than 30 may be utilized, such as for performing other medical procedures with the fluid-exchange device 100.

In some embodiments, the first needle 108 and the second needle 110 may have lengths selected for a particular medical procedure. For example, one or both shafts may have a relatively short length when the fluid-exchange device 100 is intended for use in a confined anatomical space. In some cases, one or both of the needles 108, 110 may have a length between about 0.125 inches and about 0.375 inches. For instance, the needle length may be about 0.25 inches for some embodiments configured for anterior chamber paracentesis. In some embodiments, the needle length may be longer, such as for facilitating procedures at different depths or at different surgical sites, such as joints (e.g., knee joint). For example, one or both of the needles 108, 110 may have a length between about 0.5 inches and about 3 inches. In various implementations, the first needle 108 and the second needle 110 may have the same length. In other implementations, the first needle 108 and the second needle 110 may have different lengths.

In some embodiments, the first tip 132-1 and the second tip 132-2 may be positioned relative to one another in a desired manner. For example, the first tip 132-1 and the second tip 132-2 may terminate at approximately the same axial position. In other cases, one of the first tip 132-1 and the second tip 132-2 may be slightly advanced or slightly retracted relative to the other. In some implementations, the first tip 132-1 and the second tip 132-2 may be spaced apart by a distance 140. For instance, the distance 140 may be measured tip-to-tip. In some embodiments, the distance 140 may be about 0.2 inches (about 5 mm) such as embodiments configured for anterior chamber paracentesis.

In some embodiments, the first needle 108 and the second needle 110 may extend from the same side of the dual-flow body 102. For example, FIGS. 1-1 to 1-3 illustrate an arrangement in which both the first needle 108 and the second needle 110 project outwardly from the second side 122 of the dual-flow body 102. In some cases, positioning the first needle 108 and the second needle 110 on the same side may permit the fluid-exchange device 100 to be advanced toward a surgical site as a coordinated assembly. In various embodiments, this arrangement may facilitate simultaneous or near-simultaneous placement of the first needle 108 and the second needle 110 at the surgical site, including penetrating into the surgical site.

In some embodiments, the first needle 108 and the second needle 110 may be arranged in a generally parallel relationship. For example, the shafts of the first needle 108 and the second needle 110 may extend in substantially the same direction from the dual-flow body 102. In some implementations, the first needle 108 and the second needle 110 may be coplanar. For instance, the first needle 108 and the second needle 110 may lie generally within a common plane. In various embodiments, an approximately parallel and coplanar arrangement may improve handling of the fluid-exchange device 100 and may facilitate coordinated insertion of the first needle 108 and the second needle 110 into the surgical site. In some embodiments, the first needle 108 and the second needle 110 may be coplanar and angled with respect to one another. For example, the tips 132 of first needle 108 and the second needle 110 may be angled slightly toward one another or slightly away from one another. In some cases, the first needle 108 and the second needle 110 may be angled outwardly or inwardly by up to about 15 degrees (e.g., +15 degrees to -15 degrees). In some embodiments, angling the first needle 108 and the second needle 110 toward one another may facilitate positioning the tips 132 closer together while accommodating the size or geometry of other portions of the fluid-exchange device 100, such as the first body portion 112, the second body portion 114, one or more connectors, and/or one or more needle hubs. In some implementations, a more parallel arrangement may be advantageous for penetration or insertion by reducing insertion force and improving ease of placement into associated tissue.

In some embodiments, the first needle 108 and the second needle 110 may be connected to the dual-flow body 102 in a secure and/or fixed manner. For example, the first needle 108 and the second needle 110 may be stabilized from relative movement during insertion into the surgical site. In some cases, the fixed positioning and stabilization of the first needle 108 and the second needle 110 may facilitate coordinated withdrawal of anatomical fluid and administration of replacement fluid. In various implementations, the fluid-exchange device 100, including both the first needle 108 and the second needle 110, may facilitate aspiration while reducing the need for independent manipulation of separate needles. In this way, the fluid-exchange device may provide a user with improved control over placement of the first needle 108 and the second needle 110 during a medical procedure. In some embodiments, this may facilitate improved operation, reduced damage to a patient, increased fluid retrieval, or combinations thereof.

In some embodiments, the first body portion 112 may define an angled geometry. For example, the first body portion 112 may include the first fluid inlet 116 at the first side 120 and may support the first needle 108 at the second side 122 of the dual-flow body 102. In some cases, the first fluid inlet 116 may be oriented at an angle relative to the first fluid outlet 118 and/or at an angle relative to the second fluid outlet 126. For instance, the first fluid inlet 116 may face upwardly, obliquely upwardly, or otherwise at an angle relative to another portion of the dual-flow body 102. In various implementations, the angled arrangement associated with the first body portion 112 may reduce crowding between attached components, facilitate reservoir placement, improve visibility at the surgical site, or support a gravity-assisted orientation of an attached reservoir, and combinations thereof.

In some embodiments, the angular relationship associated with the first body portion 112 may be selected based on the intended procedure and desired functionality of the fluid-exchange device 100. For example, an angle 150 may be defined between an axis of the first fluid inlet 116 and an axis of the first fluid outlet 118. In some embodiments, the angle 150 may be an upward angle. In some embodiments, the angle 150 may be defined between an axis of the first fluid inlet 116 and an axis of the second fluid outlet 126. In some cases, the angle 150 may be selected to provide sufficient clearance for a connected fluid reservoir while maintaining visibility and ease of use during a procedure. In some implementations, the angle 150 may be between about 70 degrees and about 110 degrees. For instance, an angle of about 80 degrees may be utilized for some embodiments configured for anterior chamber paracentesis. In some cases, an angle of about 90 degrees may also be utilized. In some embodiments, the angle 150 may be another angle that has at least some upward component relative to the second fluid passage 106 such that, when the fluid-exchange device 100 is positioned in the generally horizontal orientation shown, sufficient hydrostatic pressure and/or gravitational force may be present in an associated reservoir to facilitate passive flow through the first fluid passage 104. In some embodiments, an angle less than 90 degrees may be advantageous, such as to reduce obstruction of a clinician's view of the surgical site. In some embodiments, the angle 150 may be approximately 0 degrees, such that the first fluid passage 104 and the second fluid passage 106 are arranged more parallel to one another. For example, such an arrangement may be utilized with a pressurized fluid reservoir where gravity may not be needed to facilitate fluid flow through the first fluid passage 104.

In some embodiments, the first body portion 112 may further include an angular deviation in another dimension. For example, FIGS. 1-2 and 1-3 may illustrate the angle 150 in a first reference plane, or an x-y plane, while the first fluid passage 104 may additionally or alternatively be angled in another dimension, such as a z dimension into or out of the page. In some cases, this angular deviation in the z dimension may orient the first fluid passage 104 in three-dimensional space relative to the second fluid passage 106. For instance, the first fluid passage 104 may be angled in the z dimension from about 90 degrees (vertical) to about 0 degrees (horizontal into or out of the page in the z dimension) or any angle therebetween. The angling of the first fluid passage 104 in the z dimension may be in addition to, or as an alternative to, the angle 150 in the x-y plane. In this way, the first fluid passage 104 may be angled relative to the second fluid passage 106 in 3-dimensional space.

In some embodiments, the second body portion 114 may define a relatively straight geometry. For example, the second body portion 114 may include the second fluid outlet 126 at the first side 120 and may support the second needle 110 at the second side 122 of the dual-flow body 102. In some cases, the second fluid inlet 124 and the second fluid outlet 126 may be oriented generally opposite one another. For instance, the second fluid passage 106 may extend approximately linearly between the second fluid inlet 124 and the second fluid outlet 126. In some implementations, the second body portion 114 may be elongated to accommodate connection to a fluid reservoir.

In some embodiments, the dual-flow body 102 may be formed from any suitable medical-grade materials. For example, one or more portions of the dual-flow body 102 may be formed from polypropylene. In some cases, one or more components of the fluid-exchange device 100 may be formed from another suitable biocompatible polymer, metal, composite, or other material, and combinations thereof.

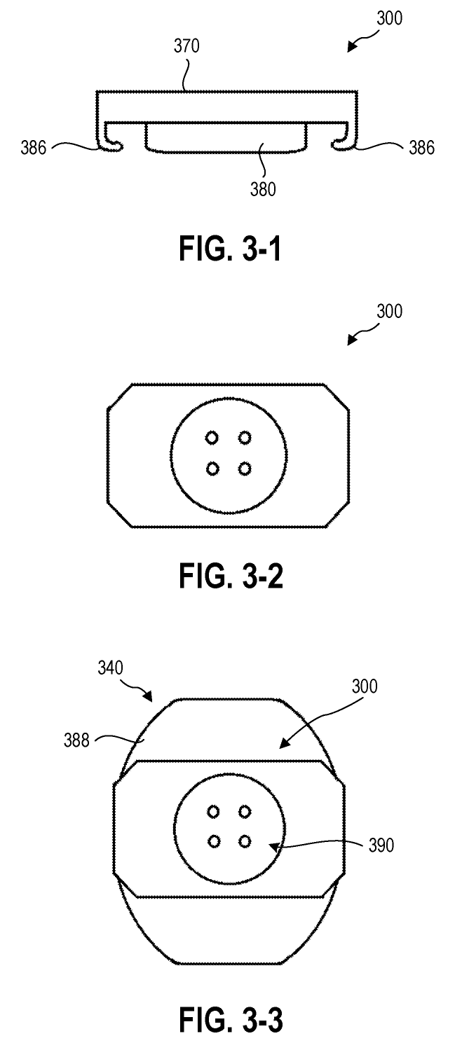

As described above, the dual-flow body 102 may be utilized in connection with one or more fluid reservoirs connected thereto. FIG. 2-1 illustrates a first side view of a fluid-exchange system 200, and FIGS. 2-2 and 2-3 illustrate second side views of the fluid-exchange system 200, according to at least one embodiment of the present disclosure. In some embodiments, the fluid-exchange system 200 may include a dual-flow body 202 in combination with a first fluid reservoir 240 and a second fluid reservoir 242 connected thereto. For example, FIG. 2-2 may illustrate the fluid-exchange system 200 in a pre-procedure or pre-aspiration state, such as with the first fluid reservoir 240 containing a replacement fluid 252 and the second fluid reservoir 242 in an initial plunger position before withdrawal of an anatomical fluid. In some cases, FIG. 2-3 may illustrate the fluid-exchange system 200 during and/or after a medical procedure, such as with the first fluid reservoir 240 at least partially depleted of the replacement fluid 252 and the second fluid reservoir 242 at least partially filled with an anatomical fluid 262 following aspiration. In some implementations, the fluid-exchange system 200 may include any one or more of the features and/or functionalities described above with respect to the fluid exchange device 100 of FIGS. 1-1-1-3.

In some embodiments, the fluid-exchange system 200 may include the first fluid reservoir 240 and the second fluid reservoir 242 connected to the dual-flow body 202. For example, the first fluid reservoir 240 may be connected in fluid communication with a first fluid passage 204, and the second fluid reservoir 242 may be connected in fluid communication with a second fluid passage 206. In some cases, the first fluid reservoir 240 may contain the replacement fluid 252 for delivery through a first flow path 205, and the second fluid reservoir 242 may be configured to receive the anatomical fluid 262 through a second flow path 207. In various implementations, the first fluid reservoir 240 and the second fluid reservoir 242 may be medical syringes. For instance, the first fluid reservoir 240 may be an influx syringe and the second fluid reservoir 242 may be an efflux syringe. In other embodiments, one or both of the first fluid reservoir 240 and the second fluid reservoir 242 may be another suitable fluid reservoir, such as a cartridge, cassette, chamber, bottle, bag, or other fluid container. In some embodiments, the first fluid reservoir 240 may have a volume selected based on the intended procedure and desired functionality of the fluid-exchange system 200. For example, the first fluid reservoir 240 may have a volume between about 0.5 mL and about 10 mL. The first fluid reservoir 240 may have a volume of about 3 mL, such as for embodiments configured for anterior chamber paracentesis. In some embodiments, the second fluid reservoir 242 may have a volume selected based on the intended procedure and desired functionality of the fluid-exchange system 200. For example, the second fluid reservoir 242 may have a volume between about 0.5 mL and about 10 mL. The second fluid reservoir 242 may have a volume of about 1 mL, such as for embodiments configured for anterior chamber paracentesis. In some embodiments, one or both of the first fluid reservoir 240 and the second fluid reservoir 242 may be connected directly to the dual-flow body 202. In other implementations, one or both of the first fluid reservoir 240 and the second fluid reservoir 242 may be connected to the dual-flow body 202 remotely, such as by medical tubing, extension tubing, a fluid line, or another conduit that maintains fluid communication with the respective fluid passage.

In some embodiments, the first fluid reservoir 240 may be connected to the dual-flow body 202 at a first fluid inlet 216. For example, the first fluid reservoir 240 may include a first reservoir body 244 and a first reservoir connector 246 configured to connect to the dual-flow body 202. In some cases, the first reservoir connector 246 may be a Luer-lock connector, a threaded connector, a friction-fit connector, a slip-fit connector, or another suitable connector. The first reservoir connector 246 may connect to a corresponding connector on the dual-flow body 202. In various implementations, the first fluid reservoir 240 may be removably connected to the dual-flow body 202. In some embodiments, the second fluid reservoir 242 may be connected to the dual-flow body 202 at a second fluid outlet 226. For instance, the second fluid reservoir 242 may include a second reservoir body 248 and a second reservoir connector 251 configured to connect to the dual-flow body 202. In some cases, the second reservoir connector 251 may be a Luer-lock connector, a threaded connector, a friction-fit connector, a slip-fit connector, or another suitable connector. The second reservoir connector 251 may connect to a corresponding connector on the dual-flow body 202. In some cases, the second reservoir connector 251 may be similar to or different than the first reservoir connector 246. In some embodiments, one or both of the first reservoir connector 246 and the second reservoir connector 251 may be indirectly connected to the dual-flow body, such as where one or both fluid reservoirs are connected remotely by tubing or another fluid conduit.

In some embodiments, the first fluid reservoir 240 may contain the replacement fluid 252, and the second fluid reservoir 242 may be positioned to receive the anatomical fluid 262. In various implementations, the fluid-exchange system 200 may be assembled, filled, primed, and/or otherwise prepared before insertion of the first needle 208 and the second needle 210 into the surgical site. In some embodiments, this initial configuration may permit a user to position the fluid-exchange system 200 for use while maintaining replacement fluid availability in the first fluid reservoir 240.

In some embodiments, the second fluid reservoir 242 may include a plunger 256. For example, the plunger 256 may be received within the reservoir body 248 and may be movable relative thereto to facilitate aspiration through the second flow path 207. In some cases, the plunger 256 may include one or more sealing surfaces configured to seal against an interior surface of the reservoir body 248. For instance, the plunger 256 may maintain a substantially fluid-tight interface with the reservoir body 248 such that withdrawal of the plunger 256 facilitates aspiration of the anatomical fluid 262 into the second fluid reservoir 242. FIG. 2-2 may illustrate the plunger 256 in an initial position, such as a depressed, inserted, or advanced position prior to aspiration. In various implementations, the second fluid reservoir 242 may initially contain little or no fluid, such as little or no anatomical fluid 262, before the system 200 is used for a medical procedure. In some embodiments, the system 200 may be used for a medical procedure by withdrawing the plunger 256 relative to the reservoir body 248, thereby drawing the anatomical fluid 262 from the surgical site through the second needle 210 and into the second fluid reservoir 242.

In some embodiments, the first fluid reservoir 240 may be arranged in a filled or charged condition prior to use of the fluid-exchange system 200 for a medical procedure. For example, the first fluid reservoir 240 may be filled with the replacement fluid 252 before the first fluid reservoir 240 is connected to the dual-flow body 202. In other implementations, the first fluid reservoir 240 may be filled after connection to the dual-flow body 202. In some cases, the first fluid reservoir 240 may be filled in a sterile manner, prefilled before delivery to a user, and/or filled immediately prior to the procedure. In some embodiments, one or more air bubbles may be removed from the first fluid reservoir 240 before the procedure begins. For instance, the first fluid reservoir 240 may be primed such that the replacement fluid 252 is present and available for delivery through the first flow path 205 when desired.

In some embodiments, the reservoir body 244 of the first fluid reservoir 240 may contain the replacement fluid 252. In some examples, the replacement fluid 252 may be balanced salt solution (BSS), saline, another medically compatible fluid, such as an ophthalmically compatible fluid, or another suitable replacement fluid, and combinations thereof, depending on the intended procedure. In various implementations, the first fluid reservoir 240 may include a cap, closure, vent structure, or another feature for maintaining the replacement fluid 252 therein. For example, as described in greater detail below, a cap may be removably connected to the first fluid reservoir 240. In some embodiments, the first fluid reservoir 240 may be vented such that pressure may be equalized and such that the replacement fluid 252 may drain passively under desired conditions. In some cases, the first fluid reservoir 240 may be oriented upwardly relative to the second fluid reservoir 242 in the configuration shown in FIG. 2-2, such that the replacement fluid 252 may be available for passive delivery.

In some embodiments, the first fluid reservoir 240 and the second fluid reservoir 242 may be oriented differently relative to one another. For example, the first fluid reservoir 240 may extend upwardly or obliquely upwardly from the dual-flow body 202, while the second fluid reservoir 242 may extend more horizontally or laterally from the dual-flow body 202. In some cases, the difference in orientation may correspond to the different geometry of the first fluid passage 204 and the second fluid passage 206, as described in connection with one or more embodiments herein. For instance, an orientation, angle, and/or geometry of the first fluid passage 204 may facilitate positioning the first fluid reservoir 240 such that the first fluid reservoir 240 has at least some upward orientation. In various implementations, the different reservoir orientations may reduce crowding between the reservoirs, improve access to the handling portions of the system, and/or improve visibility at the surgical site. In some embodiments, the upward orientation of the first fluid reservoir 240 may facilitate passive and/or gravity-assisted delivery of the replacement fluid 252.

With reference now to FIG. 2-2, this figure may represent an initial setup state before aspiration begins. For example, the first needle 208 and the second needle 210 may be inserted into a target surgical site, such as an anterior chamber of an eye, after the fluid-exchange system 200 has been placed in the pre-procedure condition shown. In some cases, the second fluid reservoir 242 may be in a ready state for aspiration, while the first fluid reservoir 240 may be in a ready state for administration of the replacement fluid 252. In various implementations, the replacement fluid 252 may remain substantially static in the first fluid reservoir 240 until aspiration begins and/or until pressure conditions permit inflow through the first flow path 205. In some embodiments, a minimal amount of replacement fluid 252 may flow out of the first fluid reservoir 240 via the first needle 208 as the system 200 is awaiting use. For example, a cap or closure associated with the first fluid reservoir 240 may include one or more features that facilitate such passive flow and/or which may prevent a significant portion of the replacement fluid 252 from flowing out of the first fluid reservoir 240.

With reference now to FIG. 2-3, this figure may illustrate the fluid-exchange system 200 during and/or after a medical procedure in which fluid transfer has occurred through the fluid-exchange system 200. For example, the second fluid reservoir 242 may contain the anatomical fluid 262 withdrawn from the surgical site, and the first fluid reservoir 240 may contain a reduced volume of the replacement fluid 252 as compared with the state shown in FIG. 2-2. In some cases, FIG. 2-3 may illustrate coordinated operation of the first flow path 205 and the second flow path 207. For instance, the reduction in the volume of replacement fluid 252 in the first fluid reservoir 240 and the increase in the volume of anatomical fluid 262 in the second fluid reservoir 242 may reflect fluid exchange through the fluid-exchange system 200 during the procedure.

In some embodiments, the plunger 256 of the second fluid reservoir 242 may be withdrawn during aspiration. For example, withdrawal of the plunger 256 relative to the reservoir body 248 may cause the anatomical fluid 262 to be drawn through the second needle 210, through the second fluid passage 206, and into the second fluid reservoir 242. In some cases, FIG. 2-3 may illustrate the plunger 256 in a retracted position relative to the state shown in FIG. 2-2. In various implementations, the amount of anatomical fluid 262 collected in the second fluid reservoir 242 may vary depending on the intended procedure, the targeted sample size, and/or one or more conditions encountered during the procedure.

In some embodiments, administration of the replacement fluid 252 through the first needle 208 may occur while aspiration through the second needle 210 is taking place. For example, administration of the replacement fluid 252 may occur simultaneously with aspiration, may overlap in time with aspiration, and/or may begin after aspiration has started. In some cases, delivery of the replacement fluid 252 through the first flow path 205 may compensate, at least in part, for removal of the anatomical fluid 262 through the second flow path 207. In various implementations, this coordinated fluid exchange may mitigate pressure drop at the surgical site, may help stabilize a target chamber, or may reduce a risk of collapse or other undesirable deformation of the chamber during aspiration, and combinations thereof.

In some embodiments, the fluid level changes shown in FIG. 2-3 may reflect operation of the fluid-exchange system 200 during the procedure. For example, as the volume of the anatomical fluid 262 in the second fluid reservoir 242 increases, the volume of the replacement fluid 252 in the first fluid reservoir 240 may decrease. In some cases, the respective fluid volumes may change at the same time, at overlapping times, and/or in a related manner based on the conditions present at the surgical site and within the fluid-exchange system 200. In some implementations, the relationship between the changing fluid volumes may vary depending on hydrostatic conditions, geometry of the fluid passages, reservoir orientation, venting characteristics, aspiration force, or other procedural variables, and combinations thereof.

In some embodiments, the coordinated fluid exchange illustrated in FIG. 2-3 may facilitate removal of a larger fluid sample than might otherwise be removed if no compensating inflow were available. For example, compensating administration of the replacement fluid 252 may permit continued (and/or increased) withdrawal of the anatomical fluid 262 while reducing excessive pressure decrease at the surgical site. In some cases, this may improve the ability to retrieve a desired fluid volume. In various implementations, the coordinated inflow and outflow may improve procedural control and may reduce a risk of tissue damage associated with excessive collapse, excessive pressure reduction, and/or loss of chamber stability.

In some embodiments, delivery of the replacement fluid 252 from the first fluid reservoir 240 may be passive. For example, the replacement fluid 252 may flow through the first flow path 205 based on gravity, hydrostatic pressure, pressure differentials, vent-controlled conditions, and/or another non-driven condition. In some cases, as the plunger 256 is withdrawn and the anatomical fluid 262 is removed from the surgical site, pressure at the surgical site may decrease. For instance, the replacement fluid 252 may naturally flow from the first fluid reservoir 240, through the first flow path 205, and through the first needle 208 into the surgical site to replace at least a portion of the removed fluid. In some embodiments, this may result in a total fluid volume within the surgical site remaining substantially constant or more nearly constant than it would otherwise remain during aspiration, which may facilitate an improved procedure. In some cases, the upward orientation of the first fluid reservoir 240 relative to the second fluid reservoir 242 may contribute to the passive delivery of the replacement fluid 252.

In some implementations, delivery of the replacement fluid 252 may be pressure-assisted. For instance, a pressurized reservoir, cassette, cartridge, integrated chamber, or other fluid source may be utilized in place of or in addition to the gravity-assisted orientation of the first fluid reservoir 240. In such embodiments, the first fluid reservoir 240 may not necessarily have an upward orientation and may instead be positioned in another orientation, such as a flatter orientation. In some embodiments, delivery of the replacement fluid 252 may be pump-driven and/or electronically controlled. For example, a pump, actuator, motorized driver, or other controllable fluid-delivery mechanism may be operatively coupled to the first fluid reservoir 240 or another fluid source to actively drive the replacement fluid 252 through the first flow path 205. In some cases, such a controlled arrangement may permit delivery of the replacement fluid 252 without relying primarily on gravity and may facilitate adjustment of flow based on a desired procedure, sensed pressure conditions, user input, or another control parameter.

In some embodiments, one or both of the first fluid reservoir 240 and the second fluid reservoir 242 may be implemented using other structures besides medical syringes. For example, the first fluid reservoir 240 may be a cartridge, cassette, bottle, bag, chamber, or integrated fluid source. In some cases, the second fluid reservoir 242 may be a syringe, a collection chamber, a vial, another fluid-collection container, and/or another suitable receptacle for withdrawn anatomical fluid 262. In various implementations, the particular structures selected for the first fluid reservoir 240 and the second fluid reservoir 242 may depend on the intended procedure, desired fluid volumes, desired pressure characteristics, manufacturing considerations, and/or sterility considerations. In some embodiments, one or both of the first fluid reservoir 240 and the second fluid reservoir 242 may be positioned remotely from the dual-flow body 202 and may be connected thereto by medical tubing or another fluid conduit, rather than being directly attached to the dual-flow body 202. In some embodiments, one or both of the first fluid reservoir 240 and the second fluid reservoir 242 may be non-removably connected to the dual-flow body 202. For example, one or both fluid reservoirs may be adhesively secured, welded, bonded, overmolded, or otherwise more permanently attached to the dual-flow body 202. In other implementations, one or both of the first fluid reservoir 240 and the second fluid reservoir 242 may be formed integrally with (a continuous body with) the dual-flow body 202, such as in a more unitized or preassembled construction.

As mentioned herein, in some embodiments, the fluid-exchange system 200 may be utilized to perform an anterior chamber paracentesis. For example, the first needle 208 and the second needle 210 may be inserted through a corneal region and into an anterior chamber of an eye. In some cases, the second needle 210 may be utilized to withdraw aqueous humor from the anterior chamber through the second flow path 207 and into the second fluid reservoir 242. In various implementations, the first needle 208 may be utilized to administer the replacement fluid 252, such as balanced salt solution, through the first flow path 205 and into the anterior chamber. In some embodiments, withdrawal of the aqueous humor and administration of the replacement fluid 252 may occur in a coordinated manner such that fluid removed from the anterior chamber is at least partially replaced as the procedure proceeds. For example, as a volume of the aqueous humor is removed, a corresponding volume of the replacement fluid 252 may passively flow from the first fluid reservoir 240 into the anterior chamber as described herein. In some cases, this coordinated flow may facilitate collection of a desired sample volume while reducing excessive chamber collapse, excessive pressure reduction, and/or other undesirable effects during the procedure.

In some embodiments, the fluid-exchange system 200 may be utilized at another surgical site besides the anterior chamber of the eye. For example, the fluid-exchange system 200 may be utilized to withdraw a fluid from another anatomical space while administering a replacement fluid to that anatomical space. In some cases, the fluid-exchange system 200 may be utilized in connection with the vitreous cavity of an eye. In some embodiments, the fluid-exchange system 200 may be utilized in connection with a joint, another chamber, another cavity, or another tissue site where fluid removal and fluid replacement may be beneficial. In various implementations, the same general process described above may be used at a different site, such as by positioning the first needle 208 and the second needle 210 at the site, penetrating to a corresponding fluid location with appropriately selected needles, withdrawing an anatomical fluid through the second flow path 207, and administering a replacement fluid through the first flow path 205. In some embodiments, the fluid-exchange system 200 may be utilized for aspiration, flushing, lavage, sampling, or another procedure involving coordinated fluid withdrawal and fluid delivery.

In some embodiments, the fluid-exchange system 200 may be operated in a reverse manner as compared to one or more embodiments herein. For example, the second fluid reservoir 242 may be preloaded with a replacement fluid and may be utilized to deliver that replacement fluid through the second flow path 207 (e.g., in an inverse direction) and into a surgical site. Additionally, fluid from the surgical site may flow through the first needle 208, through the first flow path 205 (e.g., in an inverse direction), and into the first fluid reservoir 240. In some embodiments, such flow of the anatomical fluid into the first fluid reservoir 240 may occur passively, for example, based on or in response to injection of the replacement fluid into the surgical site by the second fluid reservoir 242. For example, introduction of (e.g., injection of) the replacement fluid may increase pressure at the surgical site and thereby facilitate passive flow of the existing anatomical fluid into the first fluid reservoir 240. For instance, such an arrangement may be useful for flushing, lavage, fluid exchange, dilution, rinsing, sampling, clearing debris, and/or other procedures in which introduction of a fluid into a site may cause another fluid to exit the site. In various implementations, the same dual-flow arrangement may therefore be utilized in either direction depending on the intended procedure, the desired pressure conditions, and the fluid-management objectives at the surgical site.

FIG. 3-1 illustrates a side view of a cap 300, and FIGS. 3-2 and 3-3 illustrate top views of the cap 300, according to at least one embodiment of the present disclosure. FIG. 3-3 may illustrate the cap 300 connected to an influx fluid reservoir 340. In some embodiments, the cap 300 may be utilized with one or more of the fluid-exchange systems described herein, such as in connection with a fluid reservoir of a fluid-exchange system. For example, the cap 300 may be configured to be connected to the influx fluid reservoir 340, such as the first fluid reservoir 240 shown and described in connection with FIGS. 2-1-2-3. In some cases, the cap 300 may maintain a replacement fluid within the influx fluid reservoir 340. In some embodiments, the cap 300 may facilitate pressure equalization of and/or passive fluid delivery from the influx fluid reservoir 340.

In some embodiments, the cap 300 may include a cap body 370. For example, the cap body 370 may be a structure configured to cover, close, or partially close an opening of the influx fluid reservoir 340. In some cases, the cap body 370 may extend across at least a portion of an open end of the influx fluid reservoir 340. In some embodiments, the cap 300 may include a cap base 380. For example, the cap base 380 may extend from the cap body 370 and may be sized to fit at least partially within the open end of the influx fluid reservoir 340. In some cases, the cap base 380 may define a lower insertion portion of the cap 300 that is received within an inner volume, such as a barrel, of the influx fluid reservoir 340. In some embodiments, the cap base 380 may cooperate with the cap body 370 to position one or more vent features 390 in fluid communication with the interior volume of the influx fluid reservoir 340.

In some embodiments, the cap 300 may include one or more attachment features 386. For example, the attachment features 386 may include one or more flexible fasteners, arms, tabs, clips, catches, flanges, hooks, lips, or other retention structures positioned at one or more locations around a periphery of the cap body 370. In some cases, the attachment features 386 may be deflectable and/or compliant such that the cap 300 may be snapped into place on the influx fluid reservoir 340. In various implementations, the attachment features 386 may engage one or more outwardly extending portions of the influx fluid reservoir 340. For example, the influx fluid reservoir 340 may include one or more retention structures 388, such as one or more of a tab, a rim, a collar, a finger flange, or another retention structure associated with the influx fluid reservoir 340. In some embodiments, the attachment features 386 may be configured to securely retain the cap 300 on the influx fluid reservoir 340, while still permitting removal when desired.

In some embodiments, the cap 300 may define one or more vent features 390. For example, the vent features 390 may be one or more features through which air may pass. In some cases, the vent features 390 may extend through the cap 300, such as fluidly connecting a top surface and a bottom surface of the cap 300. In various implementations, the vent features 390 may permit air to enter the influx fluid reservoir 340 when the cap 300 is installed thereon. In some embodiments, air flow through the vent features 390 may facilitate flow of a replacement fluid from the influx fluid reservoir 340. For instance, the vent features 390 may permit air ingress while allowing the replacement fluid to remain sufficiently retained within the influx fluid reservoir 340 until desired delivery conditions are present.

In some embodiments, the vent features 390 may include one or more openings, passages, channels, perforations, holes, ports, or other vent structures. For example, the vent features 390 may include a plurality of vent features arranged in a pattern. The cap 300 may include four vent features 390 in at least one embodiment. In other implementations, the cap 300 may include a single vent feature 390 or another number of vent features 390. In various embodiments, the number, size, spacing, shape, and/or arrangement of the vent features 390 may vary depending on the intended procedure and desired functionality of the associated fluid-exchange system. For instance, the vent features 390 may be circular, elongated, slotted, polygonal, irregular, or otherwise differently shaped. In some embodiments, the vent features 390 may define a total vent area selected to permit desired air ingress while reducing undesired fluid escape from the influx fluid reservoir 340. In some cases, the total vent area (e.g., of all vent features 390) may be selected based on the size of the influx fluid reservoir 340, the volume of fluid contained therein, the intended orientation of the influx fluid reservoir 340, or the desired flow characteristics through the associated fluid path, and combinations thereof. In some embodiments, a vent feature 390 may have a size on the order of about 0.015 inches (about 0.4 mm) in at least one embodiment. In other implementations, the size of each vent feature 390, the number of vent features 390, and the total vent area may be varied together to achieve a desired balance between air ingress and fluid retention.

In some embodiments, one or more of the vent features 390 may include, define, or be associated with a vent control structure. For example, the vent control structure may include a filter membrane, a porous membrane, a hydrophobic membrane, a mesh, a porous layer, a valve element, or another structure associated with one or more of the vent features 390, and combinations thereof. In some cases, the vent control structure may permit air ingress while reducing fluid egress. In various implementations, the vent control structure may reduce contamination risk by filtering incoming air before the air reaches the interior of the influx fluid reservoir 340. In some embodiments, the vent control structure may be integrated into one or more of the vent features 390. For instance, one or more vent features 390 may include a filtering membrane positioned below one or more perforations. In some embodiments, this may be useful for providing clean, filtered, or sterile air entering the influx fluid reservoir 340.

In some embodiments, the vent features 390 may include a valve element. For example, the valve element may be a one-way air inlet valve, a check valve, an elastomeric slit valve, or another valve configured to admit air while reducing or resisting reverse flow of fluid therethrough. In some embodiments, the valve element may be formed from silicone, rubber, latex, another elastomeric material, or another suitable material. In various implementations, the valve element may be positioned at, within, or adjacent one or more of the vent features 390. In some embodiments, the cap 300 may include replaceable filter media and/or a replaceable vent insert associated with one or more of the vent features 390. In some embodiments, the valve element may be utilized instead of one or more perforations. For example, the vent features 390 may include a latex-based one-way valve in place of multiple perforations.

In some embodiments, the cap 300 may be connected to the influx fluid reservoir 340 at or near the open end of the influx fluid reservoir 340. For example, the cap 300 may be connected over the open end after the influx fluid reservoir 340 has been filled with a replacement fluid. In some cases, the cap 300 may be connected by a snap-on installation in which the attachment features 386 engage the one or more retention structures 388 of the influx fluid reservoir 340. In other implementations, the cap 300 may be connected by a retained fit, threaded connection, a friction fit, or another removable engagement. In various embodiments, FIG. 3-3 may illustrate the cap 300 aligned with the open end of the influx fluid reservoir 340 such that the vent features 390 are in fluid communication with the interior of the influx fluid reservoir 340.

In some embodiments, the cap 300 may remain attached to the influx fluid reservoir 340 while permitting pressure equalization within the influx fluid reservoir 340. For example, the cap 300 may reduce splashing and/or leakage from the influx fluid reservoir 340 while still permitting air to enter the influx fluid reservoir 340 through the vent features 390. In some embodiments, the cap 300 may facilitate passive delivery of the replacement fluid from the influx fluid reservoir 340. For example, as the replacement fluid leaves the influx fluid reservoir 340, air may enter through one or more of the vent features 390. In some cases, this may reduce or prevent formation of a vacuum within the influx fluid reservoir 340 that might otherwise resist outflow of the replacement fluid. In various implementations, the vent features 390 may therefore permit the influx fluid reservoir 340 to remain in fluid communication with ambient air while the cap 300 still functions as a closure structure. In some embodiments, this may facilitate gravity-assisted and/or pressure-responsive delivery of the replacement fluid during a medical procedure. In some cases, the vent features 390 may be selected (e.g., sized and/or equipped with features) to provide enough air ingress that gravity and/or another pressure condition may facilitate a slow desired influx of fluid while still limiting undesired dripping or leakage from the influx fluid reservoir 340.

In some embodiments, the cap 300 may be formed from any suitable material. For example, the cap body 370 and/or the cap base 380 may be formed from polyethylene, silicone, another polymeric material, another elastomeric material, another composite material, and/or another suitable material. In some cases, different portions of the cap 300 may be formed from different materials. For instance, the cap body 370 may be formed from a first material and one or more structures associated with the vent features 390 may be formed from a second material. In various implementations, the cap 300 may include one or more flexible portions to facilitate snap-fit installation and one or more rigid portions to facilitate sealing and structural support.

In some embodiments, the cap 300 may have a different configuration than that shown in FIGS. 3-1-3-3. For example, the cap 300 may be replaced by or implemented as a stopper, plug, insert, membrane assembly, threaded closure, bayonet closure, or closure structure having one or more vent features 390 associated with the influx fluid reservoir 340. In some cases, a closure structure may be inserted into the open end of the influx fluid reservoir 340 rather than attached over an outer portion of the influx fluid reservoir 340. In other implementations, one or more vent features 390 may be built into the reservoir body itself rather than provided as part of a separate cap. In this way, venting may be provided for the influx fluid reservoir 340.

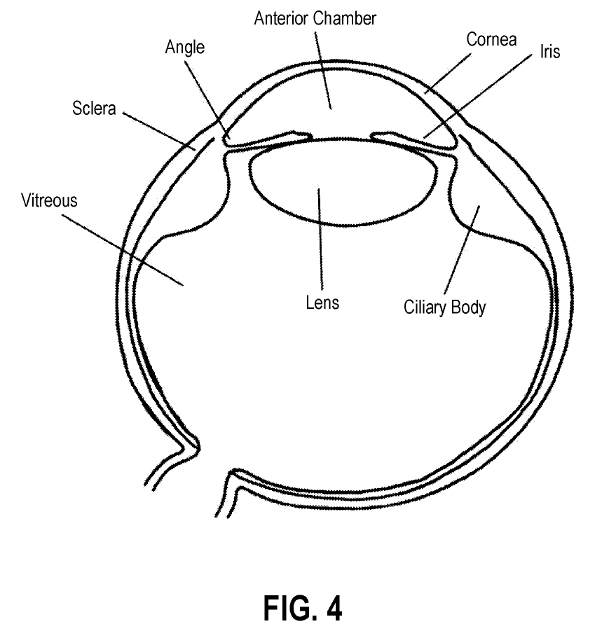

FIG. 4 illustrates an anatomical diagram of a human eye, according to at least one embodiment of the present disclosure. In some embodiments, FIG. 4 may identify one or more anatomical structures relevant to an ocular use case of the fluid-exchange systems described herein, such as the vitreous, sclera, angle, anterior chamber, cornea, iris, lens, and ciliary body. For example, FIG. 4 may be utilized to illustrate an anterior chamber paracentesis procedure in which the anterior chamber provides a target surgical site for coordinated withdrawal of an anatomical fluid and administration of a replacement fluid.

In some embodiments, the fluid-exchange system described herein may be utilized by inserting a first needle and a second needle of a fluid-exchange device through a peripheral region of the cornea and into the anterior chamber. For example, the eye may be anesthetized with a topical anesthetic and prepared with an ocular antiseptic before use of the fluid-exchange system. In some cases, the patient may be positioned in a supine position, an eyelid speculum may be placed, and the eye may be stabilized with a second instrument, such as forceps, while the needles of the fluid-exchange device are advanced into the anterior chamber. In various implementations, a dual-needle arrangement as described in one or more embodiments herein may permit insertion of the two needles in a manner similar to insertion of a single needle, while still providing increased stability within the anterior chamber.

In some embodiments, after insertion of the needles into the anterior chamber, aqueous humor may be withdrawn through an efflux fluid path while a replacement fluid, such as balanced salt solution, may be delivered through an influx fluid path. For example, before aspiration begins, positive pressure within the anterior chamber may limit or minimize influx of the replacement fluid. In some cases, withdrawal of the plunger of an efflux reservoir may lower pressure within the anterior chamber, thereby increasing passive inflow of the replacement fluid through the influx fluid path. In various implementations, this passive inflow may dampen the degree of pressure reduction within the anterior chamber, which may reduce a risk of chamber collapse and intraocular tissue injury, and may also permit a greater volume of aqueous humor to be safely withdrawn for diagnostic testing.

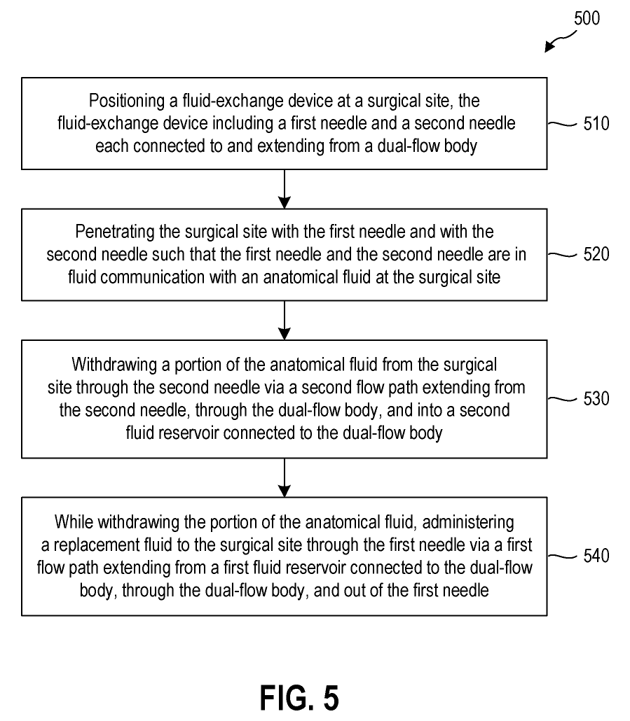

FIG. 5 illustrates a flow diagram for a method 500 or a series of acts for performing a medical procedure, according to at least one embodiment of the present disclosure. While FIG. 5 illustrates acts according to one embodiment, alternative embodiments may add to, omit, reorder, or modify any of the acts of FIG. 5.

In some embodiments, the method 500 includes an act 510 of positioning a fluid-exchange device at a surgical site, the fluid-exchange device including a first needle and a second needle each connected to and extending from a dual-flow body. In some embodiments, the method 500 includes an act 520 of penetrating the surgical site with the first needle and with the second needle such that the first needle and the second needle are in fluid communication with an anatomical fluid at the surgical site.

In some embodiments, the method 500 includes an act 530 of withdrawing a portion of the anatomical fluid from the surgical site through the second needle via a second flow path extending from the second needle, through the dual-flow body, and into a second fluid reservoir connected to the dual-flow body. In some embodiments, the method 500 includes an act 540 of while withdrawing the portion of the anatomical fluid, administering a replacement fluid to the surgical site through the first needle via a first flow path extending from a first fluid reservoir connected to the dual-flow body, through the dual-flow body, and out of the first needle.

In some embodiments, administering the replacement fluid comprises passively administering the replacement fluid based on withdrawing the portion of the anatomical fluid. In some embodiments, the first fluid reservoir is oriented upward with respect to the second fluid reservoir such that the replacement fluid is passively administered based on hydrostatic pressure and/or gravitational force. In some embodiments, the surgical site is an anterior chamber of a human eye, the anatomical fluid is an aqueous humor, the medical procedure is an anterior chamber paracentesis, and the replacement fluid is a balanced salt solution.

One or more specific embodiments of the present disclosure are described herein. These described embodiments are examples of the presently disclosed techniques. Additionally, in an effort to provide a concise description of these embodiments, not all features of an actual embodiment may be described in the specification. It should be appreciated that in the development of any such actual implementation, as in any engineering or design project, numerous embodiment-specific decisions will be made to achieve the developers’ specific goals, such as compliance with system-related and business-related constraints, which may vary from one embodiment to another. Moreover, it should be appreciated that such a development effort might be complex and time consuming, but would nevertheless be a routine undertaking of design, fabrication, and manufacture for those of ordinary skill having the benefit of this disclosure.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. For example, any element described in relation to an embodiment herein may be combinable with any element of any other embodiment described herein. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The terms “approximately,” “about,” and “substantially” as used herein represent an amount close to the stated amount that still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

What is claimed is:

1. A fluid-exchange device for a medical procedure, comprising:
a dual-flow body;
a first fluid passage formed in the dual-flow body and defined from a first fluid inlet of the dual-flow body to a first fluid outlet of the dual-flow body;
a second fluid passage formed in the dual-flow body and defined from a second fluid inlet of the dual-flow body to a second fluid outlet of the dual-flow body, wherein the first fluid inlet is oriented at an angle relative to the second fluid outlet;
a first needle connected to the first fluid outlet of the dual-flow body and extending from the first fluid passage; and
a second needle connected to the second fluid inlet of the dual-flow body and extending from the second fluid passage.

2. The fluid-exchange device of claim 1, wherein the first needle and the second needle are coplanar.

3. The fluid-exchange device of claim 1, wherein the first needle and the second needle are parallel.

4. The fluid-exchange device of claim 1, wherein the first fluid inlet is oriented at an upward angle with respect to the second fluid outlet.

5. The fluid-exchange device of claim 4, wherein the upward angle of the first fluid inlet is between about 70 degrees and about 110 degrees with respect to the second fluid outlet.

6. The fluid-exchange device of claim 1, wherein a first tip of the first needle and a second tip of the second needle are spaced apart by about 0.2 inches.

7. The fluid-exchange device of claim 1, wherein the first needle and the second needle extend from the dual-flow body on a same side of the dual-flow body.

8. The fluid-exchange device of claim 1, wherein the second fluid inlet and the second fluid outlet are oriented approximately opposite one another such that the second fluid passage extends approximately linearly through the dual-flow body.

9. The fluid-exchange device of claim 1, wherein the first fluid inlet is oriented at an angle relative to the first fluid outlet such that the first fluid passage is non-linear.

10. The fluid-exchange device of claim 1, wherein:
the first needle is part of a first needle hub, and the first needle is removably connected to the first fluid outlet of the dual-flow body by a first needle connector; and
the second needle is part of a second needle hub, and the second needle is removably connected to the second fluid inlet of the dual-flow body by a second needle connector.

11. The fluid-exchange device of claim 10, wherein the first needle connector and the second needle connector are each a Luer-lock connector.

12. The fluid-exchange device of claim 1, wherein the dual-flow body is a continuous body defining the first fluid passage and the second fluid passage.

13. A fluid-exchange system for a medical procedure, comprising:
a dual-flow body, comprising:
a first fluid passage formed in the dual-flow body and defined at least in part by a first fluid outlet of the dual-flow body; and
a second fluid passage formed in the dual-flow body and defined at least in part by a second fluid inlet of the dual-flow body;
a first needle connected to the first fluid outlet of the dual-flow body;
a second needle connected to the second fluid inlet of the dual-flow body;
a first fluid reservoir connected to the dual-flow body and in fluid communication with the first fluid passage; and
a second fluid reservoir connected to the dual-flow body and in fluid communication with the second fluid passage; wherein the first fluid reservoir is oriented at an angle with respect to the second fluid reservoir;
wherein a second flow path extends from the second needle, through the second fluid passage, and into the second fluid reservoir, and a first flow path extends from the first fluid reservoir, through the first fluid passage, and out of the first needle.

14. The fluid-exchange system of claim 13, wherein the dual-flow body further includes a first reservoir connector at a first fluid inlet of the first fluid passage, wherein the first fluid reservoir is removably connected to the dual-flow body with the first reservoir connector.

15. The fluid-exchange system of claim 14, wherein the dual-flow body further includes a second reservoir connector at a second fluid outlet of the second fluid passage, wherein the second fluid reservoir is removably connected to the dual-flow body with the second reservoir connector.

16. The fluid-exchange system of claim 15, wherein the first reservoir connector and the second reservoir connector are each a Luer-lock connector, a friction-fit connector, or a threaded connector.

17. A method of performing a medical procedure, comprising:
positioning a fluid-exchange device at a surgical site, the fluid-exchange device including a first needle and a second needle each connected to and extending from a dual-flow body;
penetrating the surgical site with the first needle and with the second needle such that the first needle and the second needle are in fluid communication with an anatomical fluid at the surgical site;
withdrawing a portion of the anatomical fluid from the surgical site through the second needle via a second flow path extending from the second needle, through the dual-flow body, and into a second fluid reservoir connected to the dual-flow body; and
while withdrawing the portion of the anatomical fluid, administering a replacement fluid to the surgical site through the first needle via a first flow path extending from a first fluid reservoir connected to the dual-flow body, through the dual-flow body, and out of the first needle.

18. The method of claim 17, wherein administering the replacement fluid comprises passively administering the replacement fluid based on withdrawing the portion of the anatomical fluid.

19. The method of claim 18, wherein the first fluid reservoir is oriented upward with respect to the second fluid reservoir such that the replacement fluid is passively administered based on hydrostatic pressure.

20. The method of claim 17, wherein the surgical site is an anterior chamber of a human eye, the anatomical fluid is an aqueous humor, the medical procedure is an anterior chamber paracentesis, and the replacement fluid is a balanced salt solution.
